(19) United States
(12) Patent Application Publication
(10) Pub. No.: US 2019/0298317 A1
(43) Pub. Date: Oct. 3, 2019

(54) INSTRUMENTED RECEPTACLE APPARATUS FOR HEALTH ANALYSIS OF BODY FLUIDS

(71) Applicant: INTERNATIONAL BUSINESS MACHINES CORPORATION, ARMONK, NY (US)

(72) Inventors: Evan G. Colgan, Yorktown Heights, NY (US); Minhua Lu, Yorktown Heights, NY (US); Vince S. Siu, Yorktown Heights, NY (US)

(21) Appl. No.: 15/943,031

(22) Filed: Apr. 2, 2018

(51) Int. Cl. A61B 10/00; A61B 5/145; A61B 5/20; G01N 33/493

(52) U.S. Cl. CPC A61B 10/0045; A61B 5/14507; A61B 5/1455; G01N 33/493; A61B 5/207

(57) ABSTRACT

A health analysis fixture includes a receptacle. A volume of water and a body fluid sample form a water line within the receptacle. A light-emitting device and a light-sensing device are disposed within the receptacle. A measurement device determines a height of the water line before and after an introduction of the body fluid sample. A computer processor is configured to receive sensor data from the light-sensing device, receive the determined height of the water line before and after the introduction of the body fluid sample from the measurement device, calculate a dilution factor for the body fluid sample from the received height of the water line before and after the introduction of the body fluid sample, receive sensor data from the light-sensing device, and interpret the received sensor data based on the dilution factor to generate health information therefrom.

### TECHNICAL FIELD

The present disclosure relates to a system for performing health analysis and, more specifically, to an instrumented receptacle including a system for performing health analysis on body fluids therein.

### DISCUSSION OF THE RELATED ART

Monitoring body fluids such as saliva, nasal mucus, tears, sputum, urine and/or sweat for biological markers and chemical composition is potentially an important tool in monitoring patient health so that diseases and other health conditions may be detected at early stages, in a cost-effective manner. Such monitoring is generally not routinely performed and is typically limited to blood/serum testing or urine testing during annual physical examinations and diagnosis of acute medical conditions.

Generally, it is desirable to minimize contact with bodily fluids and once the analysis is complete, to dispose of them in a sanitary way. Moreover, in a medical setting, the process of acquiring body fluids and performing analysis thereupon is subject to errors such as loss of samples, confusion of samples, contamination of samples, degradation of samples from sample extraction to storage, shipping and testing, improper performance of tests, improper reading of test results, etc. Additionally, the process of obtaining body fluid samples may be inconvenient, time consuming, and costly.

In part, owing to attempt to reduce error rates, the process for acquiring body fluid samples and performing analysis thereupon is rather complicated and full of steps. It is desirable to have a simple means of routinely collecting samples of various bodily fluids, testing them for biological markers and chemical composition, and disposing of them in a sanitary way with a minimum of human handling and processing.

### SUMMARY

An instrumented health analysis receptacle is configured, e.g. with sensors, to receive a volume of water and a body fluid sample mixed therein. The body fluid sample can either take the form of a fluid, such as saliva, sputum, or urine, or a tissue or other absorbent material which will readily dissolve and decompose, for collecting samples of sweat, nasal mucus, or tears. The receptacle maybe equipped to mix the body fluid sample and water together to break-up a viscous material such as sputum or mucus or to release sweat, nasal mucus, or tears from an absorbent material such as a tissue. For example, ultrasonic agitation maybe used to uniformly mix the body fluid sample and water so that the sensors are all exposed to some of the body fluid sample or its aqueous mixture. In some embodiments, the sensors, such as optical based sensors maybe placed behind a screened or filtered region of the receptacle where the pore size is selected to pass biological markers such as cells, bacteria, etc. but are too small to allow the passage of fibers that may be contained in the sample. Pore size selection may be specific to the biological marker being measured.

Once a uniform solution has been formed, the analysis may be performed. The receptacle may be equipped with a number of sensors as will be described later. If the body fluid sample was liquid only and a sufficient volume, a dilution faction may optionally be calculated. The volume of water and the body fluid sample form a water line within the receptacle. The sensors are located in the receptacle, below the water line so as to be submerged into the water and body fluid sample/mixture.

In one preferred embodiment, a light-emitting device is disposed within the receptacle, below the water line so as to be submerged into the water and body fluid sample (mixture), or in a wall of the receptacle including a (filter covered) transparent window. A light-sensing device is disposed within the receptacle, below the water line so as to be submerged into the water and body fluid sample or in the wall of the receptacle including the transparent window, spaced apart from the light-emitting device, so as to sense light emitted after passing through a portion of the sample. In one preferred embodiment, a measurement device, e.g. a level detector, determines a height of the water line before and after an introduction of the body fluid sample. A computer processor is configured to receive sensor data from the light-sensing device, receive the determined height of the water line before and after the introduction of the body fluid sample from the measurement device, calculate a dilution factor for the body fluid sample from the received height of the water line before and after the introduction of the body fluid sample, receive sensor data from the light-sensing device, and interpret the received sensor data based on the dilution factor to generate health information therefrom. In one preferred embodiment, one or more of the light-emitting and light-sensing devices can be calibrated by establishing a “base line” reading before the body fluid sample is introduced.

A method for performing health analysis optionally includes identifying a user. A body fluid sample is deposited into the receptacle to be analyzed. Volume of water present in the receptacle prior to and after the body fluid sample is added are determined. One or more tests are performed on the volume of water including a body fluid sample dissolved therein. A dilution factor of the body fluid sample is calculated based on the determined volume of water present in the receptacle prior to and after the sample addition. Results of the one or more tests may be adjusted based on the calculated dilution factor, depending on the biological marker being measured.

Selection of light-emitting devices, light-sensing devices, and/or filters that filter the light being sensed can be designed for one or more specific biological markers being measured. For example, certain biological markers can be responsive to different frequency ranges and intensities of light. Different light-emitting devices can be engaged and/or operated differently in switched sequences to do more specific biological marker detection and/or differentiation. In addition, sensors are not limited to light detections. Sensors may include an apparatus that provide one or more chemicals that react with the solution to create reactants that are measured by the sensor to infer the presence of a biological marker. In addition, sensors can include an apparatus that have chemical coatings, e.g. an antibody, that selectively reacts with a biological marker to produce a signal. A computer can operate multiple energy/chemical sources and detects, in some instances in a sequence, to create a profile of biological markers detected directly or by inference in the sample. Application of the dilution measurement can be applied on a case by case basis according to a protocol, e.g. as an analytic used as a control function in the computer.

For example, a photometric sensor based on dye or enzyme reaction with a biomarker. The concentration of the biomarker can be linked to the optic response that is either in wavelength or intensity space. However, other substances in the sample of the body fluid as well as characteristics thereof, such as pH, salt concentration, blood, cell, bacteria, and chemical or biological compounds can affect the response of the targeting biomarkers. Such effect can be studied, and an algorism can be developed based on either empirical and theoretical, or both, model to correct such interference. More than one type of sensors can be used to sensing and analyzing the sample. For example, field effect transistors (FET), bipolar junction transistors (BJT), and thin film transistors (TFT) can be used to amplify and detecting charge or potential changes due to the reaction of biomarkers with reagents. Another example is electrochemical sensor, which measures redox reactions of the biomarker and with the presence of reagents. The third example is impendence sensor which measures the impedance change due to the reaction of biomarker with reagents. Furthermore, if a reaction with biomarker can produce gas or temperature or mechanical effect, a gas, thermal, or acoustic sensor can be used. For all the sensing method mentioned interference from the other chemical substances are possible, and a computer algorithm that corrects such interference is desirable.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a schematic cross-section diagram illustrating an instrumented receptacle used for health analysis in accordance with exemplary embodiments of the present invention;

FIG. 2 is a schematic diagram illustrating the receptacle as a chemical pod or container in accordance with exemplary embodiments of the present invention;

FIG. 3A is a schematic diagram illustrating the connectivity of the instrumented receptacle used for health analysis in accordance with exemplary embodiments of the present invention;

FIG. 3B is a schematic cross-section diagram illustrating the instrumented receptacle used for health analysis in accordance with exemplary embodiments of the present invention;

FIG. 4 is a flowchart illustrating an approach for performing health analysis in accordance with exemplary embodiments of the present invention;

FIG. 5 is a flow chart illustrating an approach for performing health analysis using an instrumented receptacle used for health analysis in accordance with exemplary embodiments of the present invention; and

FIG. 6 shows an example of a computer system capable of implementing the method and apparatus according to embodiments of the present disclosure.

### DETAILED DESCRIPTION OF THE DRAWINGS

In describing exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner.

Some of the biomarkers which can be detected in body fluids currently include the following and as testing techniques improve, this list is expected to expand. For sweat, common diagnostic tests include biomarkers for cystic fibrosis, illicit drugs, electrolyte levels (e.g. sodium, chloride, potassium), ammonium, alcohols, lactate, peptides, proteins, glucose, cytokines, such as IL-6 for stress and inflammation, and dermcidin for cancer detection. For saliva, in addition to biomarkers and chemicals, it contains over 700 microorganisms that are related to oral and systemic diseases which are related to cancer, diabetics, and cardiovascular health. In tears, it has been reported that components are present that are related to various disease states such as inflammatory mediators, cytokines, growth factors, white blood cells, antigens, signaling molecules, complement components, and remodeling enzymes. For nasal mucus, biomarkers include cytokines such as IL-4, IL-6, IL-10, IFN-gamma, various bacteria such as early MRSA inflections, and TB. For sputum, a variety of cytokines markers for COPD and asthma can be detected. For urine, there are many urinary biomarkers for chronic disease monitoring including but not limited to creatinine and SDMA for chronic kidney disease monitoring, amyloid proteins in pregnant women at risk for preeclampsia, and exosomes for early cancer. For some of these biomarkers, it may not be necessary to measure a quantitative value, as detection alone can indicate that further testing is needed. Moreover, not all biological markers need to be detected with light. For example, glucose sensing may be performed electrochemically using a handheld glucometer. Ions including chloride and potassium can be detected using electrical sensors such as FET and BJTs.

Exemplary embodiments of the present invention provide an instrumented receptacle with integrated systems for performing health analysis. The receptacle fixture may be a receptacle configured to receive a body fluid sample, test the body fluid sample within the receptacle, and then dispose of the sample in a sanitary manner. The structure of the receptacle fixture may be configured to receive a body fluid sample, directly from a patient subject, without the use of intervening vessels, and accordingly, the need for obtaining body fluid samples and performing health analysis thereupon may be simplified, made more convenient, and may reduce opportunity for error. As previously mentioned, a tissue or other absorbent material which will readily dissolve and decompose, maybe used for collecting samples of sweat, nasal mucus, or tears.

The receptacle maybe equipped to mix the body fluid sample and water together to break-up a viscous material such as sputum or mucus or to release sweat, nasal mucus, or tears from an absorbent material such as a tissue. For example, ultrasonic agitation maybe used to uniformly mix the body fluid sample and water so that the sensors are all exposed to some of the body fluid sample. Some of the sensors, such as optical bases sensors which fibers from the absorbent material may interfere with maybe placed behind a screened or filtered region of the receptacle where the pore size is selected to pass fluid and biological markers such as cells, bacteria, etc. but are too small to allow the passage of fibers or tissue fragments.

Moreover, health analysis receptacle fixtures, in accordance with exemplary embodiments of the present invention, may be able to conveniently perform periodic and frequent health analysis tests so that patient health may be closely monitored, either automatically or with expert review, so that health problems may be identified at early stages.

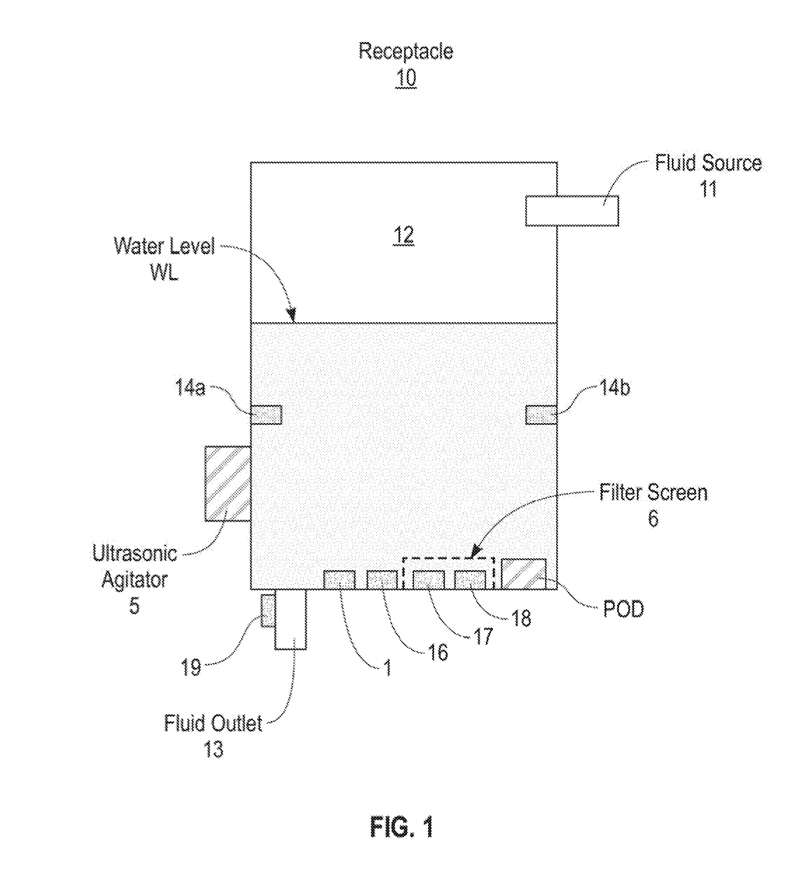

FIG. 1 is a schematic cross-section diagram illustrating a health analysis receptacle fixture in accordance with exemplary embodiments of the present invention. As can be seen from this figure, the health analysis receptacle fixture 10 may include a fluid source 11 and receptacle 12 structure with an additional one or more sensor elements 14a-19 incorporated within the receptacle 12 and a fluid outlet 13 thereof.

The receptacle 12 may contain a volume of fluid that may be introduced thereto via the fluid source 11, which may be a pipe, tube, or similar structure. The fluid may be water. For the purpose of providing a simple description, it may be assumed herein that the fluid is water, but it is to be understood that the fluid may be a fluid other than water. The fluid may be introduced into the receptacle 12 via the fluid source 11 such that the fluid does not completely fill the receptacle 12. A top surface of the fluid within the receptacle 12 may be referred to herein as a water level WL.

The receptacle maybe equipped to mix the body fluid sample and water together to break-up a viscous material such as sputum or mucus or to release sweat, nasal mucus, or tears from an absorbent material such as a tissue. For example, ultrasonic agitation or other mechanical agitation (e.g. a mixer, or vibrating holding device), 5, may be used to uniformly mix the body fluid sample and water so that the sensors are all exposed to some of the body fluid sample. Some of the sensors, 17 & 18, such as optical based sensors which fibers from the absorbent material may interfere with maybe placed behind a screened or filtered region of the receptacle, 6, where the pore size is selected to pass biological markers such as cells, bacteria, etc. but are too small to allow the passage of fibers.

The sensor elements 14a, 14b, 15, 16, 17, 18, and 19 may be disposed in the health analysis receptacle fixture 10, for example, within the receptacle 12 thereof, below the water line WL, or sealed within the wall of the receptacle 12 with transparent windows. The sensor elements 14a, 14b, 15, 16, 17, 18, and 19 may each be configured to determine a quantity (e.g. volume) of fluid that is present within the receptacle 12, both before and after an introduction of a body fluid sample, and then read chemical and physical properties of the fluid, inclusive of the body fluid sample. Thus, the patient places a body fluid sample directly into the receptacle 12 of the receptacle fixture 10 in a usual manner, without having to catch the body fluid sample in an intermediary vessel. The quantity of fluid in the receptacle 12, having been so-measured, may be used to interpret the sensor readings to correct for an extent of dilution caused by the quantity of water that was present in the receptacle 12 prior to the collection of the body fluid sample. If a tissue or absorbent material is used to transfer the body fluid sample or if the body fluid sample is small, less than 1%, compared to the initial volume of water in the receptacle, than the dilution factor would not be calculated.

The sensor elements may include an optical sensor pair that may include an optical detector/spectrometer 14a and a corresponding light source 14b. These elements may be disposed opposite to each other and facing one another within the health analysis receptacle fixture 10 and under the water line WL of the receptacle 12.

The optical sensor pair 14a and 14b may generate light from the light source 14b and the detector/spectrometer 14a may read the light having passed through the water in the receptacle 12 that is inclusive of the body fluid sample. By examining optical absorption of the light having passed through the water, given the known characteristics of the source light, the amount and type of chemicals present in the water may be understood. The detector/spectrometer 14a may also be capable of recognizing the hue of the water inclusive of the body fluid sample.

In addition to the optical sensor pair 14a and 14b, or in place of it, the health analysis receptacle fixture 10 may include an optical/RF transceiver 15, a turbidity, temperature, and conductivity sensor 16, a differential pressure sensor 17, one or more electrochemical sensors 18, and a chemical pod assembly POD, as will be described in detail below. Together, these sensors may be able to perform one or more health analysis tests such as body fluid hue testing, clarity testing, density testing, pH testing, protein testing, glucose testing, nitrite testing, leukocyte testing, ketone testing, and testing for the presence of various microscopic particles. Additional tests may include testing for the presence of pharmaceuticals, and various disease biomarkers. For example, these sensors may test for pH, glucose, protein and creatinine sensors, as well as other biomarkers such as nitrite and leukocyte.

As the aforementioned tests may be performed below the water line WL, exemplary embodiments of the present invention may use one or more sensors for appreciating the quantity of water present in the receptacle 12, both before and after the body fluid sample collection event. This may be performed by the use of flow sensors 19 that may be disposed within the fluid outlet 13, liquid level detectors, volumetric measurement sensors, weight detectors, etc. For example, the optical/RF/acoustic transceiver 15 may be disposed at a bottom of the receptacle 12 or sealed within the wall of the receptacle 12 with transparent windows to monitor the change of liquid level to measure volume and rate of the body fluid deposition, that can be used to calculate the dilution factor with respect to the known geometry of the receptacle 12. This sensor may be used in conjunction with the flow sensor 19 to monitor any release of the liquid from the receptacle 12.

The spectrometer or a camera 14a with light source 14b mounted across the water volume in the receptacle 12 may be configured to monitor a change of the hue. Hue may be determined, for example, based on spectrometer/camera data while taking into account the volume measurement and dilution factor.

The differential pressure sensor 17 may be configured to measure the pressure change and, together with the liquid level measurements, the density of the body fluid can be derived. Further, the calculated density can incorporate corrections due to the temperature of the liquid.

The turbidity, temperature, and conductivity sensor(s) 16 may be configured to measure body fluid properties such as clarity, solid content, temperature and conductivity of the body fluid. The readings of the diluted body fluid may be converted to readings for pure body fluid using the measured volumes of water, both prior to and after the introduction of the body fluid therein. Here, turbidity may be used to determine if a bacterial infection is present; e.g. the cloudier the liquid, the high number of possible pathogenic cells present.

According to some exemplary embodiments of the present invention, the health analysis receptacle 10 may be a fixture fixedly connected to a floor or wall of a room, such as by bolts, for example a toilet or urinal. According to other exemplary embodiments of the present invention, the health analysis receptacle 10 may be configured to be easily movable and/or portable. In other embodiments, the receptacle can be a test tube like device, a container used with a syringe, or a catheter tube that is used in the medical field to collect biological samples. The receptacle has a sample volume regulation. The receptacle can be made with material that can have filtering properties to filter out cell or other interference substances from the biological sample. The receptacle can be centrifuged to remove unwanted substances or concentrate certain biomarkers for detection. The receptacle can be kept at certain environment such as an incubator for bacteria or cell culture and other studies. The receptacle can be made of material that can have filtering properties for light frequency and intensity selection. Also, the receptacles can have a storage and delivery system to store and delivery reagents and diluents used for sensing. The receptacle can also have built-in electrical sensors with associated microfluidic systems if necessary for electrical sensing. The receptacle can be charged either wirelessly or by plug in. The receptacle can be placed into a reader for non-contact biomarker detections, such as optical, florescence, spectrometer, or magnetic detection. In addition, the receptacles can be stored/transported in a container/carrier that has the sensors, emitters, agitating apparatus, etc. In alternative embodiments, the receptacle can be used in water treatment facilities as a testing device to insure any water discharged meets appropriate biological and chemical standards.

According to some exemplary embodiments of the present invention, the health analysis receptacle fixture 10 may include a self-cleaning mechanism for rinsing inside surfaces of the receptacle 12 between tests. This cleaning mechanism may include evacuation of the fluid content of the receptacle 12, for example, via the outlet 13 and then the reintroduction of additional fluid, for example, through the fluid source. The fluid maybe directed especially at cleaning filter screen 6. According to one exemplary embodiment of the present invention, the reintroduction of fluid may be performed, at least in part, concurrently with the evacuation of the fluid content from the receptacle 12. In this way a rinsing function may be achieved and the surfaces of the receptacle 12, as well as the surfaces of the sensors, may be cleaned between uses to avoid cross-contamination.

As exemplary embodiments of the present invention may multiply the measured concentrations of detected substances by a dilution correction factor, during data processing, to account for the dilution that results from the presence of the fluid within the receptacle 12, it is possible that residual body fluid and other substances introduced into the receptacle 12 may contaminate future tests and the concentration of these contaminants may be multiplied during data processing. Thus, the health analysis receptacle 10 may be configured to implement a cleaning cycle which may rinse surfaces of the receptacle 12 and sensors with a cleanser and/or may agitate the surfaces of the receptacle 12 after a first testing and prior to a second testing. This cleaning operation may be in addition to the cleaning operation described above. Light may be applied to the receptacle surface with a frequency range that will sterilize/autoclave the surface.

In addition to the aforementioned sensors, which may be electronic sensors and might not rely upon the use of consumables, exemplary embodiments of the present invention may perform additional health analysis tests on the water within the receptacle 12 that contains the body fluid sample using various consumable chemical tests. These consumables may be introduced into the receptacle fixture 10 by the insertion of a chemical pod or container POD that may include a number of consumable chemicals. The pod or container POD may additionally include the aforementioned cleanser.

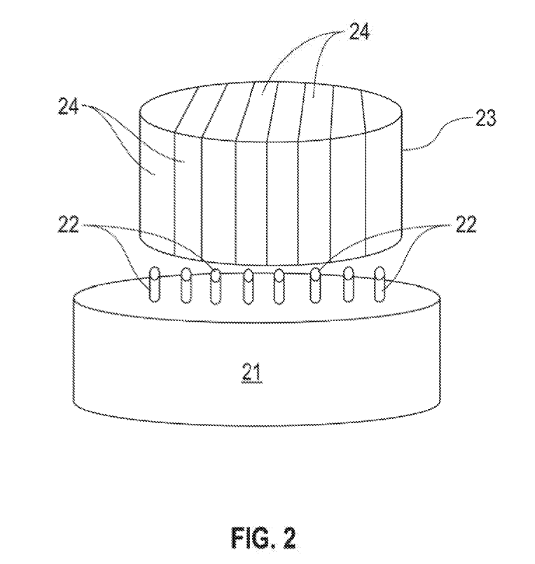

FIG. 2 is a schematic diagram illustrating the chemical pod or container in accordance with exemplary embodiments of the present invention. As can be seen from this figure, the chemical pod or container 23 may include multiple isolated compartments 24 that may each be accessed by the insertion of a connecting line/needle 22 which may pierce a cap or other surface of the chemical pod or container 23. In this way, quantities of each required testing chemical/cleansing agent may be drawn from the chemical pod or container 23, as needed, and the chemical pod or container 23 may be replaced periodically upon exhaustion in a manner similar to the way inkjet printer ink containers are changed. A receiving base unit 21 may be disposed within the receptacle fixture 10, for example, in the manner illustrated in FIG. 1. The receiving base unit 21 may include the set of connecting line/needle 22 and the tests may be performed within the receiving base unit 21 or one or more sensors connected thereto.

One type of tests that may be performed using consumables, in accordance with exemplary embodiments of the present invention, are enzyme-linked immunosorbent assay (ELISA) tests. ELISA or immunoassay procedures may be integrated inside the receptacle fixture 10 with arrays of functionalized surfaces for detection of a range of biomarkers that have suitable antibodies available such as inflammatory biomarker (e.g. interleukins, cytokines, etc.). Filtration and wash mechanisms with specialized fluids may also be drawn from the chemical pod or container 23. For ions such as sodium, potassium, etc., sensing may be performed using FETs or ion-selective electrodes. For testing for the presence of glucose or other small molecules, sensing may be performed by enzymatic reaction measured amperometrically or potentiometrically.

In this way, the health analysis receptacle 10 may be configured to perform health analysis tests on the body fluid sample dispersed into the water of the receptacle 12 using optical, fluoresces, infra-red, ultra violet, Raman spectroscopy, electrical, and/or electrochemical testing methods. The health analysis receptacle fixture 10 may also optionally be equipped with a stirring or mixing unit that agitates the water in the receptacle 12, inclusive of the body fluid sample, prior to testing so that the calculations for compensating for dilution may be simplified to assume a uniform dispersion of the body fluid sample within the receptacle of water. Alternatively, for a non-viscous fluid, it may be assumed that the force of the introduction of the body fluid sample into the receptacle 12 is sufficient to produce adequate mixing.

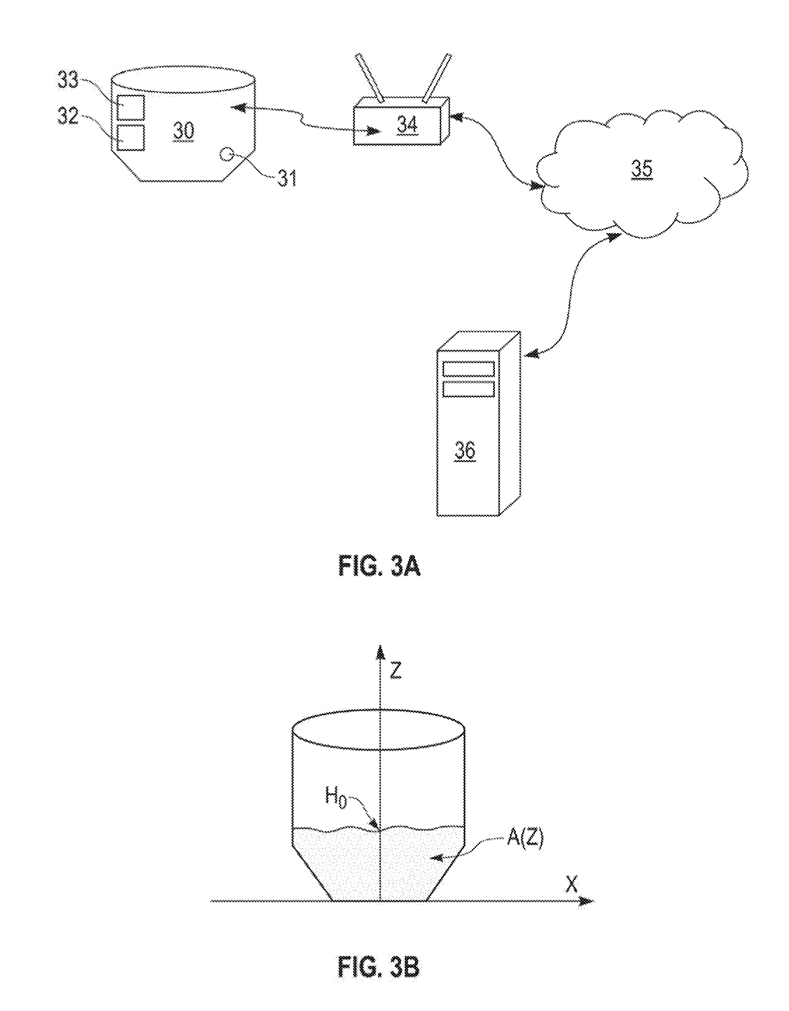

The health analysis receptacle fixtures 10, in accordance with exemplary embodiments of the present invention, may be in electronic communication with a computer system and/or cloud-based service for the periodic monitoring and reporting of health analysis results. FIG. 3A is a schematic diagram illustrating connectivity of the health analysis receptacle fixture 10 in accordance with exemplary embodiments of the present invention. The health analysis receptacle fixture 30 may include an access port 31 for inserting the chemical pod or container 23. The access port 31 may be disposed below the water line or may be above the water line and in either case, the set of connecting line/needles may serve to deliver the various chemicals from the conveniently-oriented access point to the corresponding tests which are disposed below the water line.

The health analysis receptacle 30 may further include a processor 32 which is in communication with each of the test sensors. The processor 32 may collect the test data. The processor 32 may also identify the user either actively, for example, by the user identifying him or herself to the processor 32 by an input method such as by keying in a name or ID, voice command with voice recognition, etc. Alternatively, the processor 32 may passively identify the user, for example, by determining a weight of the user that might be seated on the health analysis receptacle fixture 30 or standing on a scale disposed in front of the health analysis receptacle fixture 30, by using an RFID system, fingerprint reader, image/facial recognition, wirelessly linking to an electronic device the user carries, etc. Based on the user information, either preloaded or fetched from a remote server, personalized tests can be programmed or subscribed. In either event, the processor 32 may use a communications device 33, for example, a wireless radio, to communicate with a nearby base station 34 (e.g. a WiFi base station, a cellular base station, or another nearby device connected via Bluetooth). The base station 34 may be in communication with a server 36 over a computer network 35, such as the Internet.

Either the processor 32 or the server 36 may be responsible for calculating the undiluted test results from the diluted test results which were acquired.

The server may maintain data logs including undiluted test results, user names, and testing dates and may use this information to generate health reports and/or alerts. A health report may be a periodic report showing interpreted test results, how the interpreted test results have changed over time, and what the health implications may be. The alerts may be generated as warnings to make the user and/or a healthcare practitioner aware of a possible acute medical condition that is identified based on the health analysis results. The server may, in this way, perform long term monitoring of basic health conditions, hydration levels, chronical disease indicators, kidney health indicators, pancreatic health indicators, bladder health indicators, liver health indicators, cancer factors, and/or signs of bacterial infection.

Reports and/or alerts may also be displayed to the user, for example, using a display device associated with the health analysis receptacle fixture 30 and/or by sending a message to a personal computer and/or mobile phone of the user, which may originate from the server 36.

According to some exemplary embodiments of the present invention, the health analysis receptacle fixture 30 may include a centrifuge compartment. The water, inclusive of the body fluid sample, may be flowed into the centrifugal compartment and spun at various speeds to separate particles and sediments of different sizes. A fluidic system may be used to separate the various particle types and to transport them to an examination compartment. A low-profile microscope, incorporated therein, may be used to image the various particles. The microscope may illuminate the particles during imaging and image data may be sent to the processor 32 and/or the server 36 for analysis. A microfluidic system may then be used for further sorting, separating, and identifying biomarkers, such as DNA, microRNA, protein, exosomes, etc.

A clean and wash system, such as described above, may be used to clear contaminants from all the aforementioned elements between tests. In addition to performing cleaning of all surfaces, sensors and lines, alerts may be issued when pods need replacing and/or electrochemical sensors need replacing. Replacement may also be automatically invoked in this manner, for example, using an automated dispensing system.

As mentioned above, the processor 32 and/or the server 36 may be used for controlling and interpreting tests. This may include control of data collection, measurement processes, fluidic control and process, multiplex or parallel measurement control, etc. These functions may all be performed by a command center unit. A cognitive hub unit may collect data, process and perform initial decision making and data storage.

As described above, the health analysis receptacle fixture may determine a volume of water in the receptacle and use this to calculate a dilution of the body fluid sample if the sample is of a significant volume, such as >1% of the initial water volume. To aid in accurately calculating this value, exemplary embodiments of the present invention may also estimate a volume of body fluid introduced into the receptacle as the specimen. This may be performed, for example, by the use of sound detectors to detect a start and end of a body fluid deposit, and this time interval may be correlated and crosschecked with volume sensing. Additionally, the event duration maybe recorded and reported.

For example, volume may be calculated using known geometry of the receptacle so that total water volume is known as a function of the waterline height. The waterline height prior to use event (H₀) is read and used to calculate initial water volume (V₀). The waterline height during and after the use event is read and used to calculate body fluid volume and body fluid rate. While the initial volume V₀ represents the quantity of water diluting the body fluid sample, the difference between the final volume and the initial volume may be used to calculate the volume of the body fluid sample. This may be true as long as the outlet 13 is not passing fluid there through, as may be sensed by the flow sensor 19.

In the event that fluid is passing through the outlet 13, data from the flow sensor 19 may be used to understand and account for the loss of fluid, for example, to adjust the dilution of the sample accordingly, or simply to reject the sample.

FIG. 3B is a schematic cross-section diagram illustrating a health analysis receptacle fixture in accordance with exemplary embodiments of the present invention. The water line height may be measured, for example, using acoustic waves, electromagnetic waves, radio frequency (RF) waves, and/or optical waves. The rate of sample fluid deposition can be calculated from the change of the water line height with time, i.e., the change of the volume with time.

Thus, the volume of fluid within the receptacle 12 may be interpreted by reading the fluid height along the Z-axis H_{Z} and then plugging this value into the volume function for the receptacle 12 which is based on the known geometry thereof. The volume function (V) may be understood with respect to time, and so the volume V(z,t) may be interpreted. Time t=0 may be set as a time prior to a detection of body fluid deposition event, and after introduction of fluid into the receptacle 12 via the fluid source 11 has completed. At this time t=0, the observed initial fluid height H₀ may be used to determine the initial fluid volume V₀.

Thus, the initial water volume may be calculated as V(0)=∫₀^{H}^{₀} A(Z)dz.

Referring to FIG. 3B, the liquid volume may be measured. Assuming the height of the liquid measurement starts from the bottom of the receptacle. The cross-section area of the receptacle at height z is A(z). Default or pre-use water height is H₀ and the cross-section area is A(H₀). The initial water volume is V(0)=∫₀^{H}^{₀} A (Z)dz.

For over flow, if overflow sensor triggers at tₛₚᵢₗₗ, the over flow volume is:

Vₛₚᵢₗₗ(t)=∫ₜₛₚᵢₗₗ^{t}Q(t)dt when t>tₛₚᵢₗₗ,

where Q(t) is flow rate on overflow sensor:

Vₛₚᵢₗₗ(t)=0 when t<tₛₚᵢₗₗ.

Thus, the total volume of the body fluid at time t is V(t)=∫_{H}_{₀}^{Z(t)} A(Z)dz+Vₛₚᵢₗₗ(t). The body fluid flow rate may be calculated as R(t)=dV(t)/dt. The average flow rate R_{avg}=V(t)/T where T is the duration of the body fluid deposition.

The dilution factor, which represents an extent to which the body fluid sample is diluted by the pre-event fluid content of the receptacle, may be calculated as:

$$DF = \frac{V ( T )}{V ( 0 ) + V ( T )}$$

where the final volume after the body fluid deposition has completed is represented as V(T) or V_{T} and the initial volume is represented as V(0) or V₀.

After the dilution factor has been calculated, the dilution factor may be used to correct for the health analysis readings, where the analyte concentration is understood to be equal to the measured concentrations within the diluted sample over the dilution factor.

Density of the liquid may be measured with a pressure sensor that is installed in the receptacle, say at the bottom of the receptacle. The liquid pressure at the bottom of the receptacle is P(t)=H(t) p(t), where H(t) is the height of the liquid above sensor, and p(t) is the density of the liquid, which is the mix of water with volume V(0) and body fluid with volume of V(t). The density of the body fluid and water mixture p(t)=P(t)/H(t). The weight of the liquid in the receptacle is the sum of water and body fluid, which can be written as:

(V(t)+V₀)ρ(t)=ρ_{water}V₀+β_{body fluid} V(t);

Therefore, ρ_{body fluid}=ρ(t)+V₀/V(t)*(ρ(t)−ρ_{water}), when V(t)>0.

Using the measured liquid temperature value, the density value can be adjusted to a corresponding standard temperature value for comparison purposes.

A seat pressure sensor, located within or connected to an optional seat disposed over the health analysis receptacle fixture, may be used to determine a length of time of use of the health analysis receptacle fixture, and this data, for example, in combination with volume change information, may be used to determine an extent of user effort associated with the body fluid deposition, which may be used as data in determining urinary tract infections, and/or other health concerns.

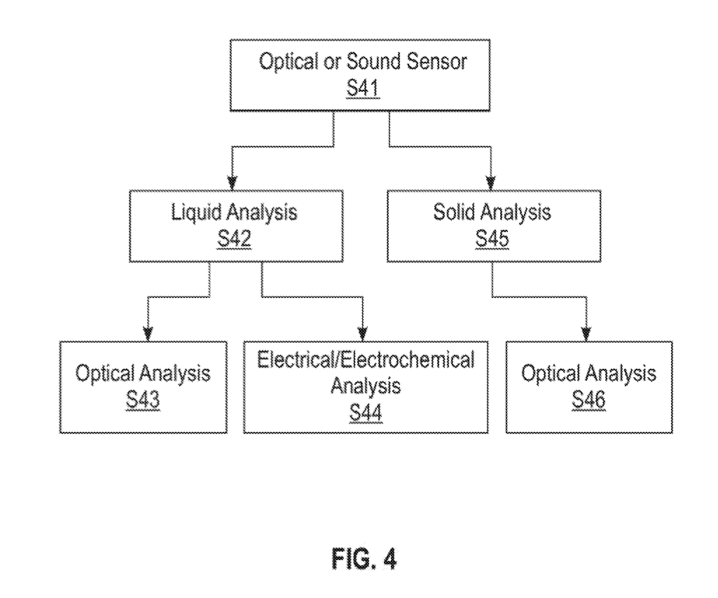

FIG. 4 is a flowchart illustrating an approach for performing health analysis in accordance with exemplary embodiments of the present invention where the health analysis receptacle has as a secondary function the ability to receive and dispose of feces. Optical, turbidity, and/or sound sensors may be used to detect an instance of use (Step S41). This data may be used to determine whether body fluid alone has entered the receptacle or if solid contaminates or solid waste is present. Where solid contaminates or solid waste is present, i.e. feces, the sample may be discarded, for example, by evacuation of the fluid volume of the receptacle, or analysis may be performed on the solid specimen (Step S45), for example, by optical analysis (Step S46).

Where solid waste is not present, body fluid analysis may be performed (Step S42).

Liquid analysis may include, for example, optical analysis (Step S43) and/or electrical/electrochemical analysis (Step S44), for example, as described above.

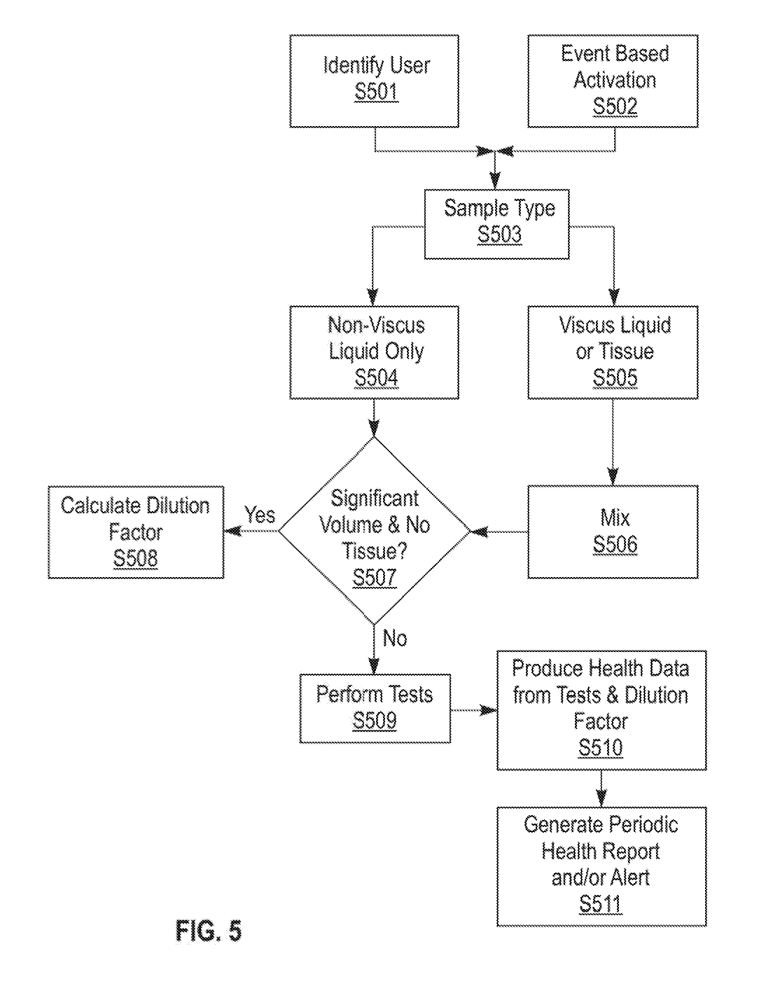

FIG. 5 is a flow chart illustrating an approach for performing health analysis using a health analysis receptacle fixture in accordance with exemplary embodiments of the present invention for a variety of bodily fluids. User identification may be performed (Step S501). As mentioned above, this may be performed either by passive sensing, such as using biometric identifiers, or by manual entry of user information. User identification may be performed either before, after, or while event-based activation occurs (Step S502). Event-based activation may be triggered by the sensing of pressure on an optional receptacle seat, for example, using the aforementioned seat pressure sensor, by picking up sound cues, or by turbulence detected in the water or by an increase in water temperature.

Next, the type of body fluid deposited is identified. S503. The non-viscous liquid only case, S504, corresponds to urine and the other body fluids such as saliva, sputum, or a tissue or absorbent material with a sample of sweat, nasal mucus, or tears are either viscous or associated with a tissue or other absorbent material, S505. For the second case, S505, agitation or mixing is applied to the receptacle to uniformly disperse the body fluid sample throughout the water in the receptacle, S506.

The volume of the body fluid sample may be determined by measuring the water volume before and after the introduction of the body fluid sample. This may include calculating the volume for the initial fluid level. This reading may be performed periodically, or it may be performed upon a sensing of a user's interaction such as the above-mentioned event-based activation. Then, sensors may be monitored to determine an end of the body fluid deposition event, after which time, a second volumetric calculation may be performed to calculate the final volume, with the change in volumes, the fluid displacement, being identified as the volume of the body fluid sample that has been introduced. If the volume of body fluid is significant, >1% of the initial fluid level, and no tissue, absorbent material or other extraneous matter was added (Step S507), a dilution factor may be calculated, S508, for example, in accordance with the formula mentioned above. The aforementioned tests may be performed using the sensors of the health analysis receptacle fixture (Step S509).

The sensor readings may then be adjusted using the optional dilution factor to produce health data (Step S510). The health data may then be used to generate periodic health reports and/or to issue a health alert, for example, when the data correlates to a potential health issue (Step S511).

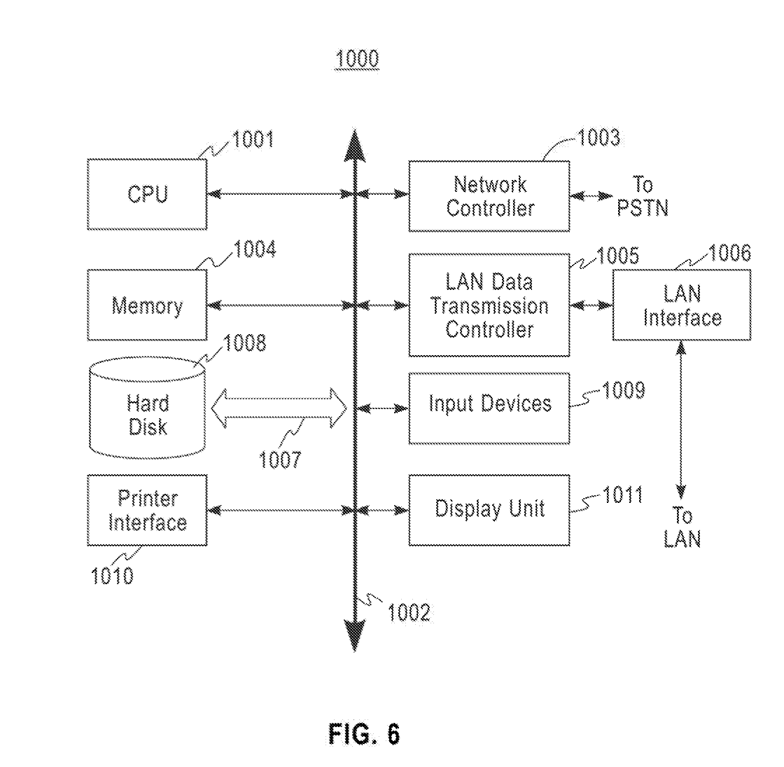

FIG. 6 shows an example of a computer system which may implement a method and system of the present disclosure. The computer system may be implemented as the processor module 32 and/or the server 36 described above. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN or wireless interface 1006, a network controller 1003, an internal bus 1002, and one or more input devices 1009, for example, a keyboard, mouse, touch input, voice and cell phone etc. As shown, the system 1000 may be connected to a data storage device, for example, a hard disk, memory devices, 1008 via a link 1007.

Exemplary embodiments described herein are illustrative, and many variations can be introduced without departing from the spirit of the disclosure or from the scope of the appended claims. For example, elements and/or features of different exemplary embodiments may be combined with each other and/or substituted for each other within the scope of this disclosure and appended claims.

What is claimed is:

1. A health analysis fixture, comprising:
a receptacle configured to receive a volume of water and a body fluid sample mixed therein, the volume of water and the body fluid sample forming a water line within the receptacle;
a mixing device such as an ultrasonic actuator for mixing fluids in the receptacle,
a light-emitting device disposed within the receptacle, below the water line so as to be submerged into the water and body fluid sample, or in a wall of the receptacle including a transparent window;
a light-sensing device disposed within the receptacle, below the water line so as to be submerged into the water and body fluid sample or in the wall of the receptacle including the transparent window, spaced apart from the light-emitting device, so as to sense light emitted therefrom;
a measurement device for determining a height of the water line before and after an introduction of the body fluid sample; and
a computer processor configured to: receive sensor data from the light-sensing device, receive the determined height of the water line before and after the introduction of the body fluid sample from the measurement device, calculate a dilution factor for the body fluid sample from the received height of the water line before and after the introduction of the body fluid sample, receive sensor data from the light-sensing device, and interpret the received sensor data based on the dilution factor to generate health information therefrom.

2. The health analysis fixture of claim 1, further comprising one or more additional sensors disposed within the receptacle, below the water line, or in the wall of the receptacle including the transparent window.

3. The health analysis fixture of claim 2, wherein the one or more additional sensors are configured to test for body fluid hue, clarity, density, pH, protein content, glucose content, nitrite content, leukocyte content, ketone content, and/or particle content.

4. The health analysis fixture of claim 2, wherein the one or more additional sensors includes an optical transceiver, a sonic transceiver, a radio frequency transceiver, a turbidity sensor, a temperature sensor, a conductivity sensor, a differential pressure sensor, and/or an electrochemical sensor.

5. The health analysis fixture of claim 2, further including a base unit configured to receive a pod having one or more chambers for storing chemicals, the base unit including one or more conduits for receiving the stored chemicals and transporting them to the one or more sensors.

6. The health analysis fixture of claim 1, further including a fluid outlet connecting the receptacle to a drain, the fluid outlet including a flow sensor for monitoring a flow of fluid out of the receptacle.

7. The health analysis fixture of claim 1, further including a communication device for connecting the computer processor to a computer server across a computer network.

8. The health analysis fixture of claim 3, further comprising a screen or filter with a maximum opening size of 0.020 mm, and preferably less than 0.010 mm.

9. The health analysis fixture of claim 1, further comprising a centrifugal compartment for spinning the water and a body fluid sample mixed therein to separate various particles therefrom and a microscope configured to image the separated particles.

10. The health analysis fixture of claim 2, further comprising a cleaning fluid dispenser configured to provide cleaning fluid to the receptacle, the light-sensing device, and/or the one or more additional sensors.

11. The health analysis fixture of claim 1, further comprising a seat disposed above the receptacle and a pressure sensor disposed therein.

12. The health analysis fixture of claim 1, further comprising a biometric identification unit configured to identify a user of the health analysis fixture.

13. A method for performing health analysis, comprising:
identifying a user;
identifying an event providing a body fluid sample to a receptacle;
determining the type of sample and mixing the body fluid and water in the receptacle if the sample or viscous or absorbed in a tissue or absorbent material,
determining a volume of water present in the receptacle prior to and after the event;
performing one or more tests on the volume of water including a body fluid sample dissolved therein;
calculating a dilution factor of the body fluid sample based on the determined volume of water present in the receptacle prior to and after the body fluid deposition event if the body fluid volume is greater than one percent of the water volume and no absorbent material is present; and
adjusting results of the one or more tests based on the calculated dilution factor if one is calculated.

14. The method of claim 13, further comprising:
measuring pressure of the water present in the receptacle prior to and after the body fluid deposition event to determine a change in pressure;
measuring a height of the water present in the receptacle prior to and after the body fluid deposition event to determine the volume of water present in the receptacle prior to and after the deposition event to determine a change in volume attributable to the body fluid sample;
measuring a temperature of the water present in the receptacle after the fluid deposition event;
calculating a density of the body fluid sample based on the change in pressure, the change in volume, and the calculated dilution factor; and
reporting the calculated density and the measured temperature.

15. The method of claim 13, further comprising transmitting the adjusted results to a server over a computer network.

16. The method of claim 13, further comprising generating a health report based on the adjusted results.

17. The method of claim 13, further comprising generating a health alert when the adjusted results are determined to be indicative of a potential health problem.

18. The method of claim 17, further comprising transmitting the health alert to a user.

19. The method of claim 13, further comprising determining when the receptacle includes solids and rejecting the adjusting results when the receptacle is determined to include solids.

20. The method of claim 13, further comprising performing a cleaning operation on the receptacle and/or one or more sensors used to perform the one or more tests after the one or more tests has been performed.
